(19) United States
(12) Patent Application Publication
(10) Pub. No.: US 2026/0207852 A1
(43) Pub. Date: Jul. 23, 2026

(54) MEANS FOR SECURING NEEDLES FOR INTERVENTIONAL MEDICAL PROCEDURES

(71) Applicant: KING FAISAL SPECIALIST HOSPITAL & RESEARCH CENTRE, RIYADH (SA)

(72) Inventors: Omar Abduljabar A. ALYAMANI, RIYADH (SA); Abdulmalik Sulaiman A. ALROBAEE, RIYADH (SA)

(21) Appl. No.: 19/032,239

(22) Filed: Jan. 20, 2025

(51) Int. Cl. A61M 5/32; A61M 25/02

(52) U.S. Cl. CPC A61M 5/32; A61M 2025/0253

(57) ABSTRACT

The application refers to a needle fixation device comprising an elongated sheet of a flexible material, wherein the elongated sheet comprises a reinforcement material having a malleable section, wherein the reinforcement material is configured such that a shape of the elongated sheet can be permanently changed by deformation of the elongated sheet at the malleable section and an adhesive layer on one side of the elongated sheet.

### TECHNICAL FIELD

The present invention is related to a means for securing/fixing needles at precise angles during medical procedures like interventional pain procedures and interventional radiology procedures.

### BACKGROUND

Interventional pain procedures involve inserting a needle into a person's tissue to reach nerves and other parts of the body. The needle, which can be inserted into a pin or holder, is usually held by the therapist's hand, which can be cumbersome. Interventional medical procedures may also involve a high risk of internal injuries and other complications if the needle is not secured properly during the procedure.

It is an object of the present invention to solve the problem of unstable holding of the needle during interventional medical procedures.

According to the present invention, the problem is solved by providing a needle fixation device comprising the features of claim 1. Embodiments of the invention are given by the dependent claims.

### SUMMARY

A needle fixation device according to the present invention, comprises an elongated sheet of a flexible material. The elongated sheet may comprise a reinforcement material having at least one malleable section. The reinforcement material is configured such that the shape of the elongated sheet can be permanently changed by deformation of the elongated sheet at the malleable section. The elongated sheet may comprise multiple pieces of one or several types of reinforcement material. The device may also comprise an adhesive layer on one side of the elongated sheet. The adhesive layer can be used for connecting the ends of the elongated sheet placed around the needle with each other thereby fixing the needle to the needle fixation device. The ends of the elongated sheet are configured such that they can be connected to a common surface such as the skin or tissue of a human with the adhesive layer on the free ends of the elongated sheet. After the needle is placed at a desired depth and angle relative to the skin or tissue of a human during a medical procedure, connecting the ends of the elongated sheet to the skin or tissue and around the needle will ensure fixation of the of needle's angle and depth with precision.

Thus, the needle fixation device according to the present invention provides an innovative solution to the challenges encountered during needle fixation in interventional pain procedures and interventional radiology procedures. The needle fixation device provides healthcare professionals with an effective means of securing needles at precise angles and depths during critical medical procedures.

According to an embodiment the elongated sheet comprises a first layer and a second layer of the flexible material and wherein the first layer and the second layer encloses the reinforcement material between them. The arrangement comprising the reinforcement material sandwiched between the first layer and the second layer of the elongated sheet of flexible material, can be held together using an adhesive. The device can comprise a further adhesive layer on one side of one of the two elongated sheets of the assembled device to connect sections of the ends of the device placed around a needle with each other and to fix the ends of the device to a surface such as a skin of a human as described above.

As an alternative the reinforcement material can be integrated or inserted into a single layer of the flexible material, or the flexible material can be molded around the reinforcement material to produce the device. The reinforcement material can be an integral part of the elongated flexible material, and a portion of the elongated sheet comprises a malleable section of the reinforcement material.

According to another embodiment the elongated sheet comprises a polyurethane film. The elongated sheet of flexible material could be made of any non-reactive medical grade materials like plastic, polyethylene or polyurethane. Other suitable materials that can be used for the elongated sheet of flexible material are polypropylene, polyvinyl chloride, cloth, foil (aluminum), fiberglass, foam or silicone rubber or combinations thereof.

According to another embodiment the reinforcement material comprises one, two or more layers, strips or rods of reinforcement material placed symmetrically about the longitudinal axis of the elongated sheet of the flexible material. According to an embodiment the reinforcement material comprises two strips that are placed parallel to each other and a distance apart from the center i.e., away from the longitudinal axis of the elongated sheet and enclosed between the first layer and the second layer of the elongated sheet of flexible material. The separation between the two strips provides a firm support along the length of the needle shaft and prevents the needle from falling/bending sideways when the needle is supported by the ends of the elongated sheet placed on a surface like skin of a patient.

According to one embodiment, instead of providing a reinforcing material in the inside of a layer of an elongated sheet of a flexible material a piece or layer of a reinforcing material is joined to a layer of an elongated sheet of a flexible material by bonding their surfaces to form the device. According to another embodiment, threads of a reinforcing material are inserted into an elongated sheet of a flexible material to form a braid or composite material.

According to a further embodiment the reinforcement material comprises a metal including one or more of stainless steel, medical grade stainless steel such as SS 304, titanium, platinum, and aluminium. Alloys comprising one or more of these and other materials are also conceivable. The reinforcing material may have deformable properties that allow the device to be shaped into the desired form when used with the hands. On the other hand, the shaping properties of the reinforcing material in the device should be such that the molded shape of the device remains stable against loads in order to fulfill the desired properties. The shaping properties of the reinforcement material can be influenced both by the material combination and by the dimensions of the pieces of reinforcement material used. This ensures a tight grip around the needle shaft and secures the needle at desired angles and depths precisely, with the help of the adhesive layer on one side of the elongated sheet.

According to yet a further embodiment the device has a flat rectangular or tape-like shape. The flat rectangular or tape-like shape ensures a tight grip around the needle shaft and formation of a stable stand or support for holding the needle when the device is placed around the needle with connecting its ends and placing the distal ends of the device on a surface such as a skin of a human.

According to another embodiment the rectangular shaped device has tapering ends. The tapered ends allow effortless application as well as removal of the device.

According to another embodiment the device has a rectangular shape at a centre section and broadens towards one or both ends. The broadened edges provide increased contact area for the elongated sheet of flexible material on a surface such as a human skin. This can enhance the fixation and stability of the needle held by the device. In a further aspect of the above embodiment, the device may have one tapered end whilst it broadens towards the other end.

According to yet another embodiment the device has a length between 100 mm and 150 mm, a width between 10 and 50 mm and a thickness between 1mm and lcm.

According to yet another embodiment the adhesive layer is covered by a removable paper. According to yet another embodiment the malleable section extends over the entire length of the reinforcement material.

According to yet another embodiment the reinforcement material extends over at least one- half, two-thirds, three-quarters or more of the length of the elongated sheet.

According to another embodiment the adhesive layer is divided into four sections by a removeable coating paper. This feature allows securing the device around a needle in at least two steps.

In a first step, the needle is inserted in the patient and adjusted at a required angle and depth with confirmation of the needle tip location in relation to its target location using imaging techniques and/or sensory stimulation testing.

In a second step, the needle is secured by pinching/pressing the malleable part of the reinforcement material around the needle shaft to have a firm hold over it and the first two sections of the adhesive layer on the respective ends of the needle fixation device placed around the needle are connected with each other by exposing the adhesive under the removable paper.

In a third step, the desired needle angle and depth are secured and fixed to the patient's skin using the two remaining adhesive sections toward the ends of the elongated sheet and away from the needle after removing their respective coating papers. This feature allows better control over the needle fixation process and thereby limiting any room for errors.

Depending on the direction of placing the strip of the needle fixation device around the needle or needle holder this procedure results in an angle formed between the direction of extension of the needle and the direction of extension of the ends of the needle fixation device having the sections of adhesive layer connected with each other. According to the various implementations of the present invention, the needle fixation device can be used to secure a needle between a maximum angle of for example 45 degrees and a minimum angle subjective to the thickness of needle shaft that allows the needle fixation device to be wrapped around it between the longitudinal extension of the needle and the direction of extension of the ends of the needle fixation device having the sections of adhesive layer connected with each other. This is just one example of how the device can be used. It is clear that other possible uses of the device are also conceivable. The sequence of the steps described can also be varied or changed.

According to another aspect a device is provided which comprises a needle or a needle holder and a needle fixation device placed around the needle such that a surface of the needle fixation device is in contact with the needle and sections of the ends of the needle fixation device placed around the needle are connected with each other so that the needle fixation device is fixed to the needle, wherein an angle is formed between the direction of extension of the needle and the direction of extension of the ends of the needle fixation device having the sections connected with each other. The needle fixation device can have some or all of the characteristics and features described above. The connection between the connected sections of the ends of the needle fixation device placed around the needle can be achieved by an adhesive such as an adhesive layer provided on the needle fixation device.

According to yet another embodiment the ends of the needle fixation device are unconnected in end sections distal to the sections of the needle fixation device connected with each other. The unconnected ends can be bent about rectangularly with respect to the direction of extension of the connected sections of the needle fixation device so that both unconnected ends extend in opposite directions and can be placed on a surface like a human skin.

The ends of the needle fixation device are configured such that they can be connected to the skin or tissue of a human with the adhesive layer. By connecting the ends of the needle fixation device to the skin or tissue of the patient the needle can be placed at a desired depth and angle with precision relative to the skin or tissue of a human during a medical procedure. Firm support for the needle fixation device over the skin during a medical procedure is provided.

According to yet another embodiment the ends of the needle fixation device distal to the sections connected with each other extend in different directions and are configured to contact a common surface. The device disclosed according to the various embodiments herein, can be used on needles over a broad thickness range (roughly from 22 gauge to 16 gauge).

### BRIEF DESCRIPTION OF DRAWINGS

Further advantages characteristics and details of the invention will result from the annexed drawing and its description in which

FIG. 1 shows components of the device according to an embodiment in an exploded view;

FIG. 2A shows the device assembly of the device according to an embodiment;

FIG. 2B shows the final assembled product;

FIG. 3 the fixation device placed around a needle in a cross-sectional view; and

FIG. 4 the fixation device placed around a needle in a perspective view.

### DETAILED DESCRIPTION

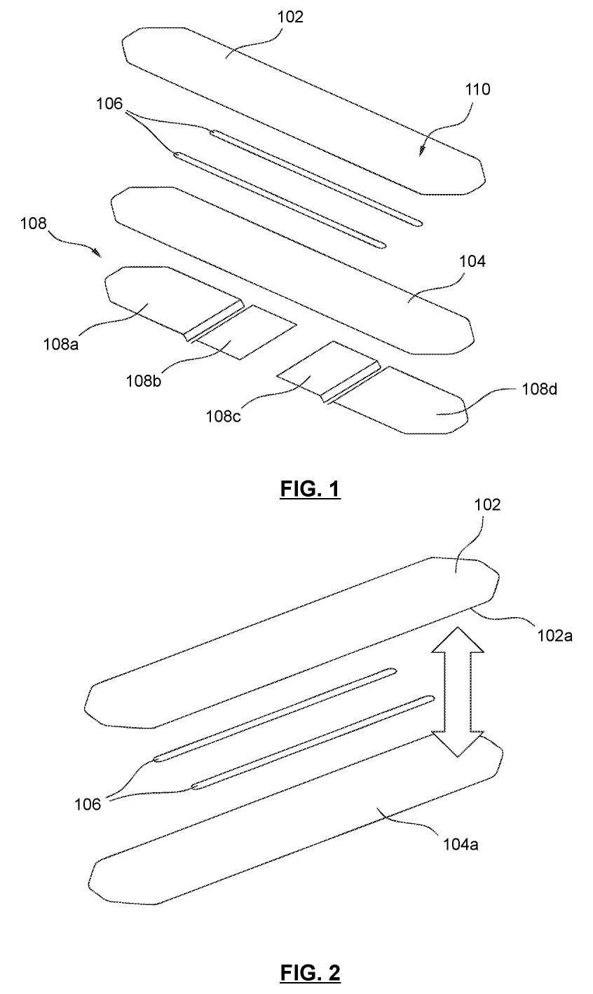

FIG. 1 shows the components of a needle fixation device 110 according to an embodiment of the present disclosure. The needle fixation device 110 comprises two elongated sheets of a flexible material, comprising a first layer 102 and a second layer 104 respectively. In the illustrated figure, the elongated sheet is comprised of polyurethane films and encased between the two elongated sheet is a reinforcement material 106. The reinforcement material 106 shown herein are two strips of medical grade stainless steel (SS 304). The reinforcement material could be any other medical grade reinforcement material with malleable properties as described above. The two layers 102 and 104, are held together by an adhesive (not shown in the figure), holding the reinforcement metal strips 106 sandwiched between them. The reinforcement metal strips are placed symmetrically along the longitudinal axis of the elongated sheets.

On one side of the device 110 is an adhesive layer (not shown in the figure). The adhesive layer is covered by a removable paper 108. The removable paper may be divided into four sections 108a-108d leaving an uncovered gap section of the needle fixation device 100 between sections 108b and 108c.

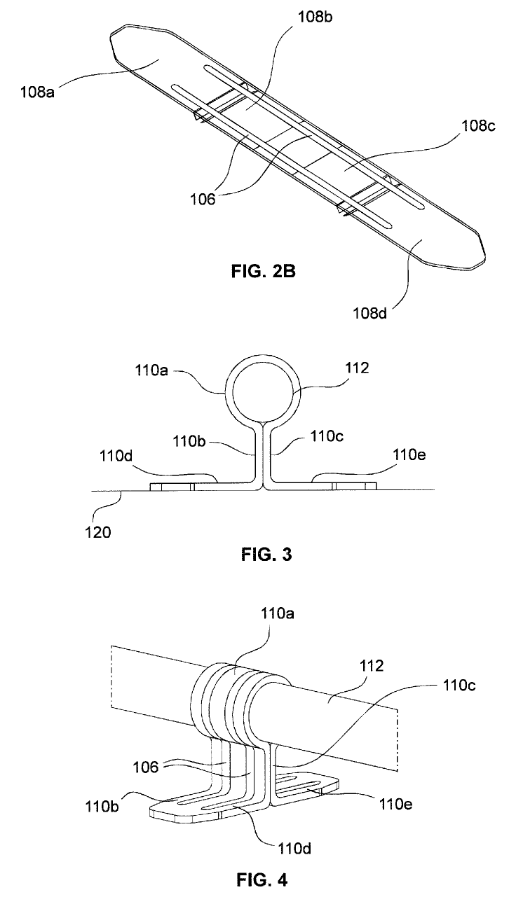

FIG. 2A shows the device assembly 200 wherein adhesive is applied on the opposite surfaces 102a and 104a of the two layers 102, 104 indicated by the arrow. The device arrangement comprising reinforcement metal strips 106 encased between the layers of elongated sheets viz., the first layer 102 and the second layer 104, are held together by the adhesive. FIG. 2B shows the final assembled product 201 after the layers 102 and 104 are connected using the adhesive.

As shown in FIG. 3 in an application the device 110 is placed around a needle or needle holder 112 such that a middle section 110a of the needle fixation device 110 with the adhesive layer is in contact with the needle 120 and sections 11 Ob and 110c of the needle fixation device 110 corresponding to sections 108b, 108c of the paper on the respective ends of the needle fixation device 100 placed around the needle 112 are brought together to connect with each other by the adhesive. The remaining sections 110d and 110e of the needle fixation device 110 corresponding to 108a and 108d sections of the paper (from FIG. 1) are further used to fix the device on to a surface (skin/body) by the adhesive sections provided thereon. To do so the remaining sections 110d and 110e of the needle fixation device 110 are bent in opposite directions out of the plane including the needle 112 and the connected sections 110b and 11 Oc of the needle fixation device 110 placed around the needle at about 90° as is shown in FIG. 4. The adhesive sections on the remaining sections 110d and 110e of the needle fixation device 110 are then secured on the skin 120 of a patient. Due to the malleable properties of the reinforcement material the needle fixation device 110 remains stable in the given shape and offers a secure support of the needle. The angle between the needle 112 and the connected sections 11 Ob and 110c of the needle fixation device 110 can be adjusted according to the requirements by placing the needle fixation device 110 at an angle around the needle 112.

Multiple modifications can be provided to the needle fixation device according to the shown embodiment without leaving the scope of the invention defined by the claims.

We claim:

1. A needle fixation device comprising:
an elongated sheet of a flexible material;
wherein the elongated sheet comprises a reinforcement material having a malleable section, wherein the reinforcement material is configured such that a shape of the elongated sheet can be permanently changed by deformation of the elongated sheet at the malleable section; and
an adhesive layer on one side of the elongated sheet.

2. The device of claim 1, wherein the elongated sheet comprises a first layer and a second layer of the flexible material and wherein the first layer and the second layer enclose the reinforcement material between them.

3. The device of claim 1, wherein the elongated sheet comprises a polyurethane film.

4. The device of claim 1, wherein the reinforcement material comprises one or more strips of material placed symmetrically about the longitudinal axis of the elongated sheet of the flexible material.

5. The device of claim 1, wherein the reinforcement material comprises one or more of stainless steel, medical grade stainless steel, titanium, platinum, and aluminium.

6. The device of claim 1, wherein the device has a flat rectangular shape.

7. The device of claim 6, wherein the rectangular shaped device has tapering ends.

8. The device of claim 6, wherein the device has a rectangular shape at a centre section and broadens towards one or both ends.

9. The device of claim 1, wherein the device has a length between 100 mm and 150 mm, a width between 10 and 50 mm and a thickness between 1mm and 1cm.

10. The device of claim 1, wherein the adhesive layer is covered by a removable paper.

11. The device of claim 1, wherein the malleable section extends over the entire length of the reinforcement material.

12. The device of claim 1, wherein the reinforcement material extends over at least one-half of the length of the elongated sheet.

13. A device comprising a needle and a needle fixation device placed around the needle such that a surface of the needle fixation device is in contact with the needle and sections of ends of the needle fixation device placed around the needle are connected with each other so that the needle fixation device is fixed to the needle, wherein an angle is formed between the direction of extension of the needle and the direction of extension of the ends of the needle fixation device having the sections connected with each other.

14. The device of claim 13, wherein the ends of the needle fixation device distal to the sections of the layer connected with each other are unconnected.

15. The device of claim 13, wherein the ends of the needle fixation device distal to the sections connected with each other extend in different directions and are configured to contact a common surface.

16. The device of claim 4, wherein the reinforcement material comprises two or more strips of material placed symmetrically about the longitudinal axis of the elongated sheet of the flexible material.

17. The device of claim 12, wherein the reinforcement material extends over at least two-thirds of the length of the elongated sheet.

18. The device of claim 12, wherein the reinforcement material extends over at least three-quarters of the length of the elongated sheet.
